(19) United States
(12) Patent Application Publication
(10) Pub. No.: US 2026/0224871 A1
(43) Pub. Date: Aug. 6, 2026

(54) DEVICES AND METHODS FOR DELIVERING A DRUG VIA AN API-LOADED TISSUE PENETRATOR

(71) Applicant: JANSSEN BIOTECH, INC., Horsham, PA (US)

(72) Inventors: PEYTON HOPSON, JACKSONVILLE, FL (US); STEPHEN G. GARA, SOUDERTON, PA (US); DRAKE SMALLEY, JACKSONVILLE, FL (US)

(21) Appl. No.: 19/152,081

(22) PCT Filed: Feb. 2, 2024
(86) PCT No.: PCT/US2024/014126

§ 371 Date: Jul. 30, 2025

Related U.S. Application Data
(60) Provisional application No. 63/483,240, filed on Feb. 3, 2023.

(51) Int. Cl. A61M 37/00; A61M 31/00

(52) U.S. Cl. CPC A61M 37/0015; A61M 31/002; A61M 2037/0023; A61M 2037/0046; A61M 2037/0061; A61M 2210/1053

(57) ABSTRACT

A drug delivery device includes at least one tissue penetrating member configured to embed into tissue, the at least one tissue penetrating member including: a plurality of cavities, and at least one payload comprising at least one active pharmaceutical ingredient (API), the at least one payload loaded into the plurality of cavities so that the at least one API can be absorbed into the tissue when the at least one tissue penetrating member is embedded in the tissue, wherein a ratio of a surface area of the plurality of cavities to a volume of the plurality of cavities is at least 0.5:1

### CROSS-REFERENCE TO RELATED APPLICATIONS

This application claims the benefit of U.S. Provisional Application No. 63/483,240, filed Feb. 3, 2023, the entire contents of which are hereby incorporated by reference herein.

### FIELD

The present disclosure relates generally to drug delivery devices, and more particularly, to drug delivery devices in which an API is loaded onto a needle.

### BACKGROUND

Microneedles, and other tissue penetrating devices, have been traditionally manufactured in monolithic-type structures with any modification from that design dependent on secondary processing (e.g., lathing, laser cutting, water jetting, etc.) to produce features in the monolithic-type structures. These features can be used to carry a specific drug payload or provide sampling ports for fluidic diagnostic devices. These features are typically recessed from the monolithic structure as the secondary processing is subtractive in nature. Additionally, features that can be fabricated before secondary processing are limited by current tissue penetrating device manufacturing methods. Typical manufacturing methods include molding (e.g., cast molding, injection molding, loss wax molding, etc.), lathing, and/or extrusion processing. Each of these methods limit the ability to have undercut features, negative draft angle structures, and internal channels.

Based on conventionally used manufacturing techniques, flexibility in loading the drug payload is limited by cavity feature size and/or balance of mechanical properties and drug active pharmaceutical ingredient (API) properties (i.e., retaining tissue penetration strength with needles comprised of excipient and drug blend). In case of cavity feature size, there is typically minimal contact area between the drug payload and needle device. The role of excipient for both adherence to the needle and toughness limits the choice and drug loading capability (i.e., increased excipient to drug ratio).

### SUMMARY

Tissue penetrating drug delivery devices include a plurality of cavities that can be loaded with a payload for delivery of one or more APIs to tissue. The plurality of cavities can be configured to provide a desired release profile for the payload (i.e., excipient(s) and API) and/or to increase the drug payload. The plurality of cavities can be configured to provide a relatively high payload-to-cavity contact area, which enhances the retention of the payload in the cavities, allowing for a lower proportion of excipient to be used in the payload and, thereby, increasing the amount of API that can be loaded in the tissue penetrating device.

According to an aspect, a drug delivery device includes at least one tissue penetrating member configured to embed into tissue, the at least one tissue penetrating member including: a plurality of cavities, and at least one payload comprising at least one active pharmaceutical ingredient (API), the at least one payload loaded into the plurality of cavities so that the at least one API can be absorbed into the tissue when the at least one tissue penetrating member is embedded in the tissue, wherein a ratio of a surface area of the plurality of cavities to a volume of the plurality of cavities is at least 0.5:1.

The plurality of cavities may be arrayed around a longitudinal axis of the at least one tissue penetrating member. The at least one payload may include an excipient, and a ratio of the at least one API to the excipient in the payload may be at least 2:1.

The at least one tissue penetrating member may be 3D printed. The at least one tissue penetrating member may be 3D printed using stereolithography or material jetting. The at least one payload may be 3D printed into the plurality of cavities.

The at least one tissue penetrating member may include a pointed tip for piercing the tissue.

The at least one tissue penetrating member may have an outer diameter of up to 2 millimeters. The at least one payload may have a total volume of at least 2 cubic millimeters. A total tissue-contacting surface area of the at least one payload may be at least 20 square millimeters.

The at least one tissue penetrating member may be configured to penetrate into a stomach wall. The drug delivery device may be configured for oral administration.

The at least one tissue penetrating member may include a plurality of microfluidic channels for holding at least a portion of the at least one payload.

The drug delivery device may have a plurality of different APIs loaded into the plurality of cavities

According to an aspect, a method of delivering at least one API to tissue includes using any of the above drug delivery devices. For example, the method may include embedding the at least one tissue penetrating member into the tissue so that the at least one API can be absorbed into the tissue.

### BRIEF DESCRIPTION OF THE FIGURES

The invention will now be described, by way of example only, with reference to the accompanying drawings, in which:

FIGS. 1A and 1B illustrate an exemplary tissue penetrator that includes a plurality of cavities for loading with one or more APIs;

FIG. 2 is an example of a conventional tissue penetrating member configuration;

FIG. 3 illustrates an example of a tissue penetrator that comprises a plurality of microfluidic channels;

FIG. 4A and FIG. 4B are cross sections of a portion of the tissue penetrator 300 that illustrate examples of possible configurations of microfluidic channels;

FIG. 5 illustrates an example of 3D printing of one or more tissue penetrators;

FIG. 6 illustrates an example of the 3D printing of a tissue penetrator in which a payload is 3D printed into the cavities of the tissue penetrator;

FIG. 7 illustrates an example of an oral delivery device that includes at least one tissue penetrator; and

FIG. 8 illustrates an example of a microneedle device that includes a plurality of tissue penetrators that extend from a substrate.

### DETAILED DESCRIPTION

Described herein are drug delivery devices that include tissue penetrators that can embed in tissue to deliver one or more APIs to the tissue. The tissue penetrators can include a plurality of cavities that can be loaded with payload that includes one or more APIs that absorb into tissue when the tissue penetrators are embedded in the tissue. The configuration of the plurality of cavities can be selected for achieving different release profiles and/or increased drug payload.

According to various embodiments, the plurality of cavities can be configured to maximize the API-to-excipient ratio in the payload. The plurality of cavities can be configured to have a relative large amount of contact area with payload loaded into the cavities. With this large amount of contact area, there is a reduced burden on the excipient to provide adhesive forces, meaning that a given volume of payload can have a higher API-to-excipient ratio, effectively increasing the API loading capacity of the tissue penetrator.

Reference will now be made in detail to implementations and embodiments of various aspects and variations of devices, systems and methods described herein. Although several exemplary variations of the devices, systems and methods are described herein, other variations of the devices, systems and methods may include aspects of the devices, systems and methods described herein combined in any suitable manner having combinations of all or some of the aspects described.

In the following description, it is to be understood that the singular forms “a,”“ ” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

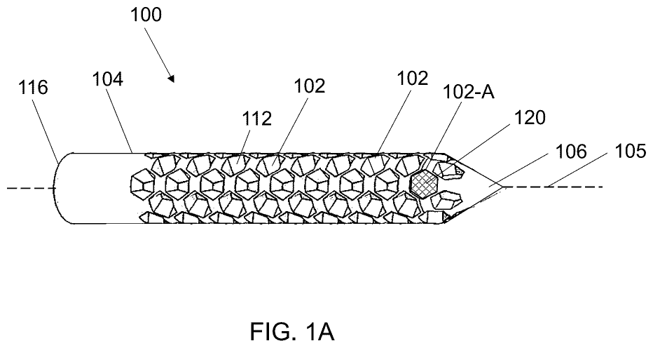

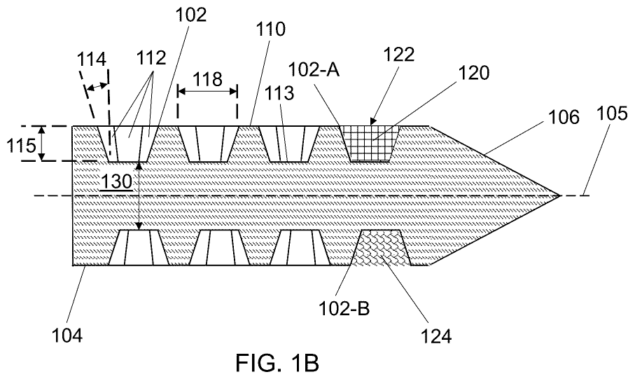

FIGS. 1A and 1B illustrate an exemplary tissue penetrator 100 that includes a plurality of cavities 102 for loading with one or more payloads that include one or more APIs. The tissue penetrator 100 is configured to penetrate a surface of tissue and embed into the tissue. When embedded in the tissue, API loaded in the cavities 102 absorbs into the surrounding tissue. As explained further below, the configuration of the cavities 102 provides a high payload-to-penetrator contact surface area, which better retains the payload in the cavities, requiring less excipient and greater relative proportion of API in the payload.

The tissue penetrator 100 includes a body 104 into which the cavities 102 are formed. A tip 106 can be configured for penetrating into tissue, such as into the skin or stomach lining. The tip 106 can be conical in shape, as illustrated, or can include one or more bevels that form a sharp pointed tip. The body 104 can be straight as illustrated or can be curved or otherwise angled. The body 104 can have one or more barbs or other projecting features that help retain the tissue penetrator in the tissue. The proximal end 116 of the body 104 can be attached to or configured for attachment to a main body (not shown) of a drug delivery device, such as an intra-organ drug delivery device or an orthopedic implant. A plurality of tissue penetrators 100 can be mounted to a substrate, such as a transdermal patch or a surgical mesh, for simultaneously delivering one or more APIs at multiple locations.

The plurality of cavities 102 are formed in the body 104 and are arrayed around the longitudinal axis 105 of the tissue penetrator 100. Payload can be loaded into the cavities 102 for delivery to the tissue. The payload can include one or more APIs and, optionally, excipient. In FIGS. 1A and 1B, one of the cavities—cavity 102-A—is shown loaded with payload 120.

The configuration of the cavities 102 can be tailored for the desired volume of payload and exposed surface area of the payload, factors which affect the amount of payload available for absorbing into the tissue and the rate of that absorption. With reference to the cross-section of FIG. 1B, the volume of each cavity 102 is defined by the area 118 of the cavity 102 at the surface 110 of the body 104 (which defines the exposed surface area of the payload that can contact tissue when the payload is loaded into the cavity 102), the depth 115 of the cavity 102 from the surface 110 of the body 104, and any draft angle 114 of the walls 112 of the cavity 102. The walls 112 can be straight or have a positive or negative draft angle. In the illustrated example, the walls 112 have a positive draft angle 114. The depth 115 of the cavity 102 can be less than a radius of the body 104. The depth 115 of the cavity 102 may be selected to provide a sufficient volume of the payload while still providing sufficient diametric material thickness 130 of the body 104 between cavities 102 such that the body 104 has sufficient strength to withstand penetrating forces.

The shape, size, and number of cavities 102 can be tailored for a given application to provide a desired release profile and/or payload volume. Cavity configuration can be adjusted to adjust the payload exposed surface area (the surface area of the payload that is exposed to surrounding tissue—indicated by reference numeral 122 in FIG. 1B) to volume ratio. A higher surface area to volume ratio can provide a faster release rate because more of the payload can be in contact with the surrounding tissue. A higher surface area to volume ratio can be achieved, for example, via a greater number of shallower cavities.

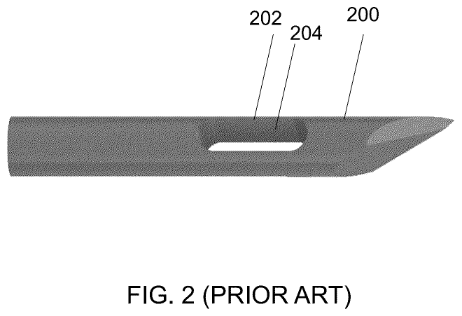

Another advantage that can be provided by the cavities 102 of tissue penetrator 100 is an increase in the drug to excipient ratio relative to conventional tissue penetrating members. An example of a conventional tissue penetrating member configuration is illustrated in FIG. 2. The conventional tissue penetrating member 200 includes a single large cavity 202 for loading payload. Comparing tissue penetrating member 100 to conventional tissue penetrating member 200, the same cavity volume (and, thereby, payload volume) can be achieved but with much higher contact surface area between the surfaces of the cavity 102 and the payload. In other words, the relatively larger number of smaller cavities 102 of tissue penetrator 100 provide more contact area between the payload and the cavity walls 112 than the single large cavity 202 of the conventional tissue penetrating member 200. This greater area of contact between the tissue penetrator and payload leads to a reduced need for the excipient to provide adhesive forces for retaining the payload to the tissue penetrator 100. As the contacting surface area increases, an effective payload exposed surface area (exposed surface area of the payload—e.g., indicated by reference numeral 122 in FIG. 1B) that is equivalent to the effective payload exposed surface area of the conventional tissue penetrating member 200 can be achieved with a reduction in the excipient volume fraction. This relatively lower required volume fraction of excipient allows for greater amounts of API for the same payload volume relative to the conventional tissue penetrating member 200.

An illustrative example of the relatively higher payload-to-cavity contact area provided by the tissue penetrator 100 of FIG. 1 relative to the conventional tissue penetrating member 200 of FIG. 2 can be demonstrated by configuring both designs to have the same total cavity volume of 2.5 mm³. The contact area between the walls 204 of the cavity 202 of the conventional tissue penetrating member 200 and a payload loaded in the cavity 202 is about 0.13 mm². In contrast, the total contact area between the walls 112 and bases 113 of the cavities 102 of the tissue penetrator 100 and payload loaded in the cavities 102 is about 11.72 mm². Thus, the tissue penetrator 100 can be configured to provide around two orders of magnitude greater payload-to-cavity contact area relative to a conventional design for the same payload volume. With these exemplary comparative configurations and assuming the conventional single cavity design requires a conventional 1:1 API-to-excipient ratio for retention in the cavity, the tissue penetrator 100 can provide the same payload volume at a greater than 9:1 API-to-excipient ratio, which means that the tissue penetrator 100 can be loaded with nine times the amount of API as the conventional design for the same payload volume. This increase in API loading capacity can be achieved without excessively increasing the payload exposed surface area (the surface area of the payload that can be in contact with the tissue) and, thus, without excessively affecting the absorption rate. For example, in the example where the conventional design and the tissue penetrator 100 are configured for a total cavity volume of 2.5 mm³, the payload exposed surface area is about 22.45 mm² for the conventional tissue penetrating member 200 and about 26.35 mm² for the tissue penetrator 100.

Another advantage that can be provided by the multiple cavities 102 of tissue penetrator 100 is that different APIs can be loaded into the same tissue penetrator. In other words, with reference to FIG. 1B, a first cavity 102-A can be loaded with a payload 120 that has a first API or combination of APIs and a second cavity 102-B can be loaded with a different payload 124 that has a second API or combination of APIs that is different than the first.

It will be understood to a person having ordinary skill in the art that the configuration of the cavities described above with respect to FIGS. 1A and 1B is merely exemplary and that the cavities can be configured differently for different applications to achieve the desired balance between payload volume, payload exposed surface area, and payload-to-penetrator contact area. Tissue penetrators can be configured to provide a ratio of cavity surface area to cavity volume of at least 0.5:1, at least 1:1, at least 1.5:1, at least 2:1, at least 2.5:1, at least 3:1, at least 3.5:1, or at least 4:1.

With the relatively higher cavity surface area to cavity volume ratio of the penetrators described herein, suitable payloads can be loaded to the penetrators with an API to excipient ratio of at least 1:1, at least 2:1, at least 3:1, at least 4:1, at least 5:1, at least 6:1, at least 7:1, at least 8:1, at least 9:1, or at least 10:1. Penetrators can be configured for payloads having an API to excipient ratio of at most 100:1, at most 50:1, at most 20:1, at most 15:1, or at most 10:1.

Although cavities 102 of FIGS. 1A and 1B are illustrated as having a hexagonal shape with sides that have positive draft angles, this shape is merely exemplary and it should be understood that the cavities can be any shape, such as cylindrical, conical, cubic, slot-shaped, or irregularly shaped, and can have straight sides, curved sides, sides with negative draft angles, and any combinations thereof. Cavities can be elongate—i.e., having their major dimension extend in the direction of with the longitudinal axis 105 of the penetrator 100—or can extend circumferentially around the longitudinal axis 105.

In some embodiments, the plurality of cavities can be or include a plurality of microfluidic channels. According to various embodiments, microfluidic channels can be used in concert with osmotic materials to generate pressure gradients for delivery of a payload to tissue. The microfluidic channels can be used for fluid ingress and/or API egress that can be tuned for each specific payload. Additionally, microfluidic channels can be used when extended API release time is desired.

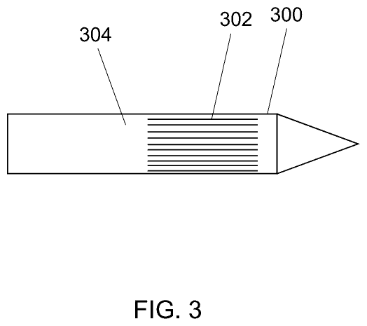

FIG. 3 illustrates an example of a tissue penetrator 300 that comprises a plurality of microfluidic channels 302 formed in rows that extend longitudinally on the surface of the body 304 of the tissue penetrator 300. This configuration of microfluidic channels is merely exemplary, and it will be understood to one of skill in the art that any desirable arrangement of microfluidic channels can be formed in the body 304.

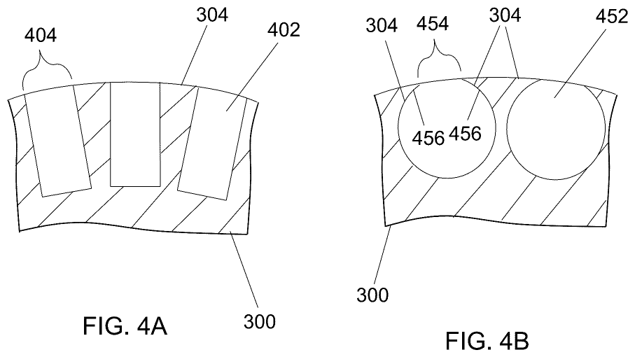

FIG. 4A and FIG. 4B are cross sections of a portion of the tissue penetrator 300 of FIG. 3 illustrating examples of possible configurations of microfluidic channels, such as microfluidic channels 302 of FIG. 3. The microfluidic channels 402 of FIG. 4A are in the shape of straight-sided grooves. The microfluidic channels 452 of FIG. 4B are in the shape of cylindrical grooves, which form undercuts 456 beneath the surface 430 of the tissue penetrator 300. The size of the openings 404 and 454 of the microfluidic channels 402 and 452, respectively, can be selected to achieve the desired payload-to-tissue contact area. Because of the undercut 456, the cylindrical microfluidic channels 452 can provide a greater volume for the same size opening relative to the straight-sided microfluidic channels 402.

Tissue penetrators can have microfluidic channels of uniform size and/or shape or microfluidic channels of varying size and/or shape. Sizes of microfluidic channels can range in size from microns in cross-sectional width up to 500 microns or larger in cross-sectional width. In some embodiments, microfluidic channels are formed during 3D printing of the tissue penetrator. Microfluidic channels can extend the entire length of a tissue penetrator or may extend only a portion of the length of the tissue penetrator. Microfluidic channels can be long relative to their width or diameter, such as having an aspect ratio of 500:1 or more, or can be short relative to their width or diameter, such as having an aspect ratio around 1:1.

In some embodiments, a tissue penetrator may include multiple different cavity configurations, such as to accommodate different types of payloads. For example, a tissue penetrator may have smaller cavities, such as a smaller cavity volume or area 118, for loading with a first payload and larger cavities, such as a larger cavity volume or area 118, for loading with a second payload that is different than the first. This arrangement can provide for delivery of different APIs, different quantity of APIs, and/or different release rates of APIs with the same tissue penetrator.

Tissue penetrators can be sized according to a given application, such as for achieving a desired penetration depth and/or for achieving a desired total payload volume. For example, a plurality of relatively small tissue penetrators, often referred to as microneedles, can be mounted to a patch and pressed into the skin for API delivery into the skin, such as beneath the stratum corneum, relatively larger tissue penetrators can be built into oral delivery devices for embedding into the stomach lining, and still larger tissue penetrators can be configured for orthopedic application in which the tissue penetrators embed into bone. Tissue penetrators can have a range of different diameters. For example, tissue penetrators can have diameters that correspond with diameters of standard hypodermic needle gauges. For example, a tissue penetrator can have a diameter corresponding to hypodermic needle gauge of 7, 8, 9, 10, 11, 12, 13, 14, 15, 16, 17, 18, 19, 20, 21, 22, 23, 24, 25, 26, 27, 28, 29, 30, 31, 32, 33, or 34, corresponding to an outside diameter of about 4.57 mm, 4.19 mm, 3.76 mm, 3.40 mm, 3.05 mm, 2.77 mm, 2.41 mm, 2.11 mm, 1.83 mm, 1.65 mm, 1.47 mm, 1.27 mm, 1.07 mm, 0.91 mm, 0.82 mm, 0.72 mm, 0.64 mm, 0.57 mm, 0.51 mm, 0.46 mm, 0.41 mm, 0.36 mm, 0.34 mm, 0.31 mm, 0.26 mm, 0.24 mm, 0.21 mm, or 0.18 mm, respectively. Accordingly, tissue penetrators can have an outer diameter of up to 5 mm, such as up to 4.5 mm, up to 4 mm, up to 3.5 mm, up to 3 mm, up to 2.5 mm, up to 2 mm, up to 1.5 mm, up to 1 mm, or up to 0.5 mm. Tissue penetrator lengths (as measured from a distal tip to a proximal end that is attached or attachable to a support structure) can be less than 20 mm, less than 15 mm, less than 10 mm, less than 5 mm, less than 1 mm, or less than 0.5 mm. Tissue penetrator lengths can be at least 0.1 mm, at least 0.5 mm, at least 1 mm, at least 5 mm, or at least 10 mm.

The size of the tissue penetrator and number and size of cavities of the tissue penetrator can be selected to achieve a total cavity volume that provides for a desired total volume of payload. For example, the tissue penetrator can be configured for a total payload volume of at least 0.5 mm³, at least 1 mm³, at least 1.5 mm³, at least 2 mm³, at least 2.5 mm³, at least 3 mm³, at least 3.5 mm³, at least 4 mm³, at least 4.5 mm³, or at least 5 mm³. The tissue penetrator can be configured for a total payload volume of no more than 10 mm³, no more than 8 mm³, no more than 6 mm³, or no more than 4 mm³.

The cavities of tissue penetrators can be configured to provide a total payload-to-tissue contact area desired for a given application. As noted above, the total payload-to-tissue contact area can be tuned to achieve a desired API release profile. Total payload-to-tissue contact area can be at least 1 mm², at least 5 mm², at least 10 mm², at least 15 mm², at least 20 mm², at least 30 mm², or at least 50 mm². Total payload-to-tissue contact area can be at most 100 mm², at most 50 mm², at most 30 mm², at most 20 mm², or at most 10 mm².

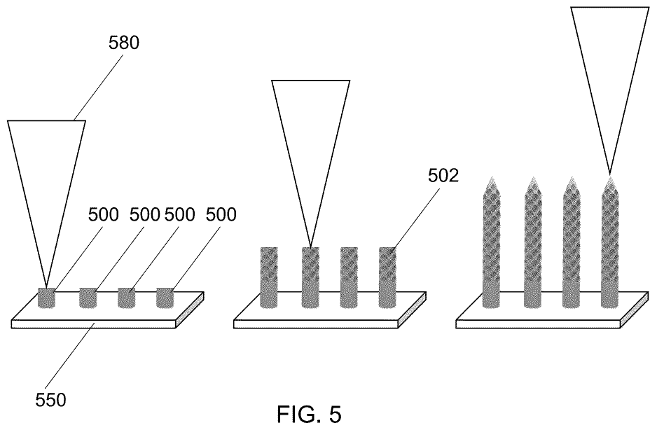

According to various embodiments, tissue penetrators are made using one or more additive manufacturing processes. For example, with reference to FIG. 5, one or more tissue penetrators 500 can be built up on a substrate 550 using a 3D printing system 580. Suitable 3D printing systems can include stereolithography, material jetting systems, binder jet systems, and powder bed fusion systems. The cavities 502 can be formed by the additive manufacturing process, which can allow for the formation of a much greater range of shapes and sizes of cavities than would be achievable or practical using other manufacturing techniques, such as subtractive manufacturing techniques or molding techniques. For example, undercut features, cavities that are interconnected beneath the surface of the tissue penetrator, and/or microfluidic channels are features that may be formed in the tissue penetrators using additive manufacturing that may not be possible using other manufacturing techniques.

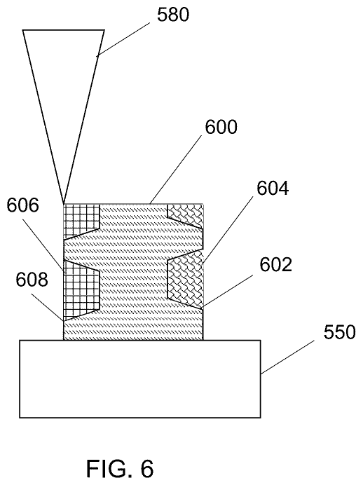

In some embodiments, the payload is formed into the cavities during the additive manufacturing process. FIG. 6 illustrates an example of the 3D printing of a tissue penetrator 600 in which a payload 604 is 3D printed into the cavities 602 simultaneously with the formation of the cavities 602. 3D printing of payloads can allow for different types of payloads to be deposited in different cavities of the same tissue penetrator. For example, payload 604 can be 3D printed into a first set of cavities and a different type of payload 606 can be 3D printed into a second set of cavities 608.

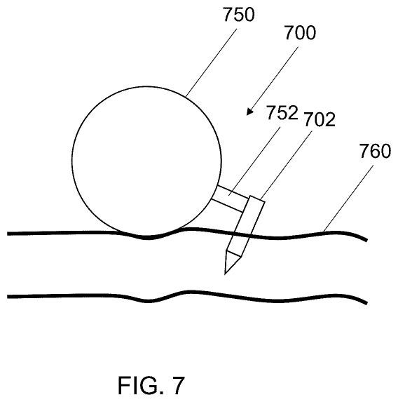

As noted above, various embodiments of tissue penetrators can be incorporated into various drug delivery devices for a variety of different applications. FIG. 7 illustrates an example of an oral delivery device 700 that includes at least one tissue penetrator 702 for delivering one or more APIs to tissue 760 of the digestive tract, such as to the stomach lining. The oral delivery device 700 can include a main body 750 to which the tissue penetrator 702 is connected. The main body 750 can be configured for oral administration and to be conveyed by the digestive tract to a desired location where the tissue penetrator is forced into the tissue. In some embodiments, the main body 750 includes a mechanical actuator 752 that forces the tissue penetrator 702 into the tissue—for example, driven by a spring positioned within main body 750. In some embodiments, the tissue penetrator 702 is stored within the main body 750 and deployed at a desired time or upon reaching a desired location. For example, the main body 750 may include a dissolvable catch that when dissolved via interaction with stomach acid releases an actuator that deploys one or more tissue penetrators. The tissue penetrator 702 may be configured to passively fall out of the tissue after a period of time or may be configured to dissolve over a period of time.

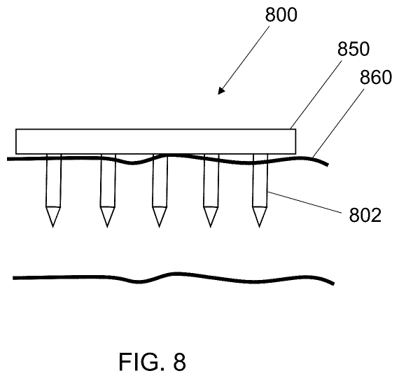

FIG. 8 illustrates an example of a microneedle device 800 that includes a plurality of tissue penetrators 802 that extend from a substrate 850 for embedding into tissue 860. The microneedle device 800 can be, for example, a patch, an orthoplate, or a hydrogel. The device 800 can be, for example, a patch that is pressed onto a patient's skin to deliver one or more API's loaded in the plurality of tissue penetrators beneath the skin surface. The patch can be manually removed after a sufficient period of time has passed for the one or more APIs to be absorbed into the tissue.

The tissue penetrator could be incorporated into a surgical staple, such as incorporated into or forming the penetrating ends of the surgical staple. The tissue penetrator could be configured to carry an API designed to enhance wound closure and healing. The tissue penetrator may be loaded to a device (e.g., a handheld device) that forces the tissue penetrator into tissue, such as via spring action. For example, a user may position a delivery end of the device at a desired location on a patient and may actuate the device (such as via a button push or trigger pull) and the device may force the tissue penetrator into the tissue to a desired depth.

The tissue penetrator could be (or could be incorporated into) an implantable rod for oncology treatment. The tissue penetrator could be (or could be incorporated into) orthopedic screws, femoral nails, and/or tendon anchors.

In some embodiments, tissue penetrator can be made of (or include) a metal, a ceramic material, or a polymeric material. The tissue penetrator material can be (or include) silicon or a metal or metal alloy such as stainless steel, titanium, magnesium allows, or a nickel titanium alloy. Exemplary types of medical grade polymeric materials include polycarbonate, liquid crystalline polymer (LCP), polyether ether ketone (PEEK), cyclic olefin copolymer (COC), and polybutylene terephthalate (PBT).

In some embodiments, the tissue penetrator material can be (or include) a biodegradable polymeric material. Exemplary types of medical grade biodegradable materials include polylactic acid (PLA), polyglycolic acid (PGA), PGA and PLA copolymer, and polyester-amide polymer (PEA).

In some embodiments, the tissue penetrator material can be (or include) an absorbable polyurethane, polycaprolactone (PCL), polydioxanone (PDO), polypropylene fumarate (PPF), poly(trimethylene carbonate) (PTMC), combinations thereof, and copolymers thereof with PLA and/or PGA.

In some embodiments, the tissue penetrator material can be (or include) photocurable resins composed of (meth)acrylate terminated absorbable polyester oligomers.

In some embodiments, the tissue penetrator or a portion thereof can be made from a dissolvable or degradable material. A dissolvable or degradable material can be any solid material that dissolves or degrades during use. For example, a tissue penetrator may be made to dissolve or degrade sufficiently in the tissue into which it is embedded. In some embodiments, the dissolvable or degradable material is selected from a carbohydrate or a sugar. In some embodiments, the dissolvable or degradable material is polyvinyl pyrrolidone (PVP). In some embodiments, the dissolvable or degradable material is selected from the group consisting of hyaluronic acid, carboxymethylcellulose, hydroxypropylmethylcellulose, methylcellulose, polyvinyl alcohol, sucrose, glucose, dextran, trehalose, maltodextrin, and any combination thereof.

In some embodiments, the tissue penetrator or a portion thereof may include an imaging agent for enabling visualization of the tissue penetrator by an imaging system, which can be useful for confirming placement of the tissue penetrator in applications in which the tissue penetrator penetrates tissue within the body. The imaging agent can be, for example, a contrast agent that can be detecting by a fluoroscopic imaging system. In some embodiments, the imaging agent is a component of a material that forms at least a portion of the body 104 of tissue penetrator 100 of FIG. 1A. For example, the imaging agent may be a component of a 3D printing material used to 3D print the tissue penetrator. Additionally, or alternatively, the imaging agent can be loaded into one or more cavities of the tissue penetrator. For example, the imaging agent can be loaded into a set of one or more cavities and payload with one or more APIs can be loaded into a different set of one or more cavities, which can be done using a 3D printing process.

Although tissue penetrating devices are described above for delivering an API into tissue, tissue penetrating devices can be configured with a plurality of cavities, according to the principles described herein, for taking samples from tissue. For example, a tissue penetrating device with unfilled cavities can insert into tissue, and cells, fluid, and/or other substance present in the tissue may migrate into the cavities. The tissue penetrating device can then be extracted from the tissue and the sample used, such as for diagnostic purposes.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A drug delivery device comprising:
at least one tissue penetrating member configured to embed into tissue, the at least one tissue penetrating member comprising:
a plurality of cavities, and
at least one payload comprising at least one active pharmaceutical ingredient (API), the at least one payload loaded into the plurality of cavities so that the at least one API can be absorbed into the tissue when the at least one tissue penetrating member is embedded in the tissue,
wherein a ratio of a surface area of the plurality of cavities to a volume of the plurality of cavities is at least 0.5:1.

2. The drug delivery device of claim 1, wherein the plurality of cavities are arrayed around a longitudinal axis of the at least one tissue penetrating member.

3. The drug delivery device of claim 1, wherein the at least one payload comprises an excipient, and a ratio of the at least one API to the excipient in the payload is at least 2:1.

4. The drug delivery device of claim 1, wherein the at least one tissue penetrating member is 3D printed.

5. The drug delivery device of claim 4, wherein the at least one tissue penetrating member is 3D printed using stereolithography or material jetting.

6. The drug delivery device of claim 1, wherein the at least one payload is 3D printed into the plurality of cavities.

7. The drug delivery device of claim 1, wherein the at least one tissue penetrating member comprises a pointed tip for piercing the tissue.

8. The drug delivery device of claim 1, wherein the at least one tissue penetrating member has an outer diameter of up to 2 millimeters.

9. The drug delivery device of claim 1, wherein the at least one payload has a total volume of at least 2 cubic millimeters.

10. The drug delivery device of claim 9, wherein a total tissue-contacting surface area of the at least one payload is at least 20 square millimeters.

11. The drug delivery device of claim 1, wherein the tissue is a stomach wall.

12. The drug delivery device of claim 1, wherein the drug delivery device is configured for oral administration.

13. The drug delivery device of claim 1, wherein the at least one tissue penetrating member comprises a plurality of microfluidic channels for holding at least a portion of the at least one payload.

14. The drug delivery device of claim 1, wherein a plurality of different APIs are loaded into the plurality of cavities.

15. A method of delivering at least one API to tissue using the drug delivery device of claim 1, the method comprising: embedding the at least one tissue penetrating member into the tissue so that the at least one API can be absorbed into the tissue.
